(19) United States
(12) Patent Application Publication
(10) Pub. No.: US 2013/0253290 A1
(43) Pub. Date: Sep. 26, 2013

(54) PARAMAGNETIC MATERIAL PATCH

(71) Applicant: Systagenix Wound Management Ltd., North Yorkshire (GB)

(72) Inventors: Samit Kumar Gupta, Bel Air, MD (US); Sophia Elizabeth Berger, Palo Alto, CA (US); April Dutta, Milpitas, CA (US)

(73) Assignee: Systagenix Wound Management Ltd., North Yorkshire (GB)

(21) Appl. No.: 13/897,856

(22) Filed: May 20, 2013

Related U.S. Application Data
(63) Continuation-in-part of application No. 13/481,545, filed on May 25, 2012.
(60) Provisional application No. 61/490,944, filed on May 27, 2011.

(51) Int. Cl. A61B 5/055

(52) U.S. Cl. CPC A61B 5/055
USPC 600/309

(57) ABSTRACT

A microneedle patch for assessing oxygenation at a localized region of interest includes microneedle projections which extend into a region of interest, a layer of material bonded to the microneedles which allows the patch to releasably engage the localized region of interest, and a paramagenetic material dispersed within the patch. An oxygen impermeable layer of material may be disposed over the oxygen permeable layer of material such that environmental oxygen is inhibited from interacting with the paramagnetic material.

### CROSS-REFERENCE

This application is a Continuation in Part of U.S. patent application Ser. No. 13/481,545 filed May 25, 2012; which claims the benefit to U.S. patent application Ser. No. 61/490,944 filed May 27, 2011; the entire contents of both related applications are incorporated herein by reference. All publications, patents, and patent applications mentioned in this specification are herein incorporated by reference to the same extent as if each individual publication, patent, or patent application was specifically and individually indicated to be incorporated by reference.

### STATEMENT REGARDING FEDERALLY SPONSORED RESEARCH OR DEVELOPMENT

Not applicable.

### REFERENCE TO A “SEQUENCE LISTING”

Not applicable.

### BACKGROUND OF THE INVENTION

### Field of the Invention

The present invention generally relates to systems and methods for assessment of oxygenation. More particularly, the present invention relates to paramagnetic material patch systems and methods of use in the assessment of oxygen tensions in biological systems such as wounds or organs in humans or animals, or in other regions of the body. Use of paramagnetic material patch systems preferably uses electron paramagnetic resonance (EPR) oximetry. Measurement may be conducted in any region of the body, including on the skin (cutaneous) or below the skin (subcutaneous).

Oxygen is a fundamental nutrient in the body which influences virtually every physiological process including metabolism, growth, and tissue repair. Measurement of oxygen tension has tremendous value in a number of biological settings. Thus, measurement of the partial pressure of oxygen, pO₂ is useful in evaluating and understanding many physiological, pathological, and therapeutic processes. In particular, measurement of oxygen tension is an important parameter in providing therapy such as for treatment of wounds, as well as in diagnostic procedures.

There are a number of existing systems and methods for assessing oxygen in tissue. Some of these include the Clark electrode, fluorescence quenching, oxygen binding to myoglobin and hemoglobin, chemiluminescence, phosphoresence quenching, and spin label oximetry. However, few systems and methods exist for assessing oxygen tension in tissue due to challenges with accuracy, repeatability, portability, and simplicity.

More recently, the use of electron paramagnetic resonance (EPR) techniques have been proposed for measurement of oxygen tension. EPR is disclosed in greater detail in U.S. Pat. Nos. 5,494,030; 5,706,805; and 5,833,601; the entire contents of which are incorporated herein by reference. Electron Paramagnetic Resonance (EPR) oximetry is a technique for measuring oxygen tension. The principle of EPR oximetry is based upon the Zeeman effect, as well as spectral broadening of absorption spectra of paramagnetic probe materials. EPR paramagnetic probe materials have unpaired electrons whose spins align with an externally applied magnetic field. Upon excitation by microwave energy, these electrons move to a higher energy state before relaxing back to the lower energy state. In the presence of oxygen molecules (which are also paramagnetic) the molecular relaxation of these paramagnetic molecules is affected in a way which causes the EPR spectrum to broaden. This broadening of the spectrum provides an indication of oxygen tension.

Diagnosis and treatment of chronic wounds and other conditions requires a reliable assessment of oxygen tension, as opposed to other metrics of tissue oxygenation such as oxygen saturation. There are a number of issues today which limit the ability of clinicians to assess oxygen tension reliably, accurately, and quickly. A major limiting factor which has prevented accurate measurement of oxygen tension is the presence of gas barrier layers on the surface of tissue, such as the stratum corneum on skin. These barrier layers make it difficult to reliably assess tissue oxygen from the surface of a tissue. The most common solution for overcoming this challenge is to heat the tissue. However, this confounds the measurement by artificially raising the measured oxygen value and adds complexity to the measurement and system. A measurement apparatus which can cross tissue barriers and simply provide accurate oxygen measurements would therefore be desirable. Such probes would preferably be low cost to manufacture, provide accurate results, and may be sterile and disposable.

It would therefore be desirable to provide an improved EPR probe for assessment of oxygenation which overcomes some of the challenges described above, thus enabling broader use of this important technology. It would also be desirable to provide improved methods that enable the direct measurement of localized oxygen concentration in a human or animal subject. At least some of these objectives will be met by the present disclosure.

### BRIEF SUMMARY OF THE INVENTION

The present invention generally relates to systems and methods for assessment of oxygenation. More particularly, the present invention relates to paramagnetic microneedle patch systems and methods for measuring oxygen tensions in biological systems such as wounds or organs in humans or animals, or in other regions of the body. Measurement of oxygen tension preferably utilizes electron paramagnetic resonance (EPR) oximetry. Measurement may be conducted in any region of the body, including on the skin (cutaneous) or below the skin (subcutaneous).

In a first aspect of the present invention, a microneedle patch for assessing oxygenation at a localized region of interest comprises microneedle projections that extend into the region of interest, a layer of material bonded to the microneedle projections which enable the assembly to releasably engage the localized region of interest and a paramagenetic material disposed within the patch. Another embodiment is identical to the one just described but also includes an additional layer of material which is disposed over the microneedles such that environmental oxygen is inhibited from interacting with the paramagnetic material. The paramagnetic material may be disposed anywhere within the microneedle patch including in the microneedles and/or the layer bonded to the microneedles. The microneedles may be made from an oxygen permeable material such as polydimethylsiloxane or polymethylmethacrylate. Other materials may be used to make the microneedles such as silicon and SU-8.

In another aspect of the present invention, a method for assessing oxygenation at a localized region of interest comprises providing a microneedle patch having paramagnetic material dispersed in a layer thereof and releasably coupling the patch with the localized region of interest. Oxygenation at the localized region of interest is then assessed using electron paramagnetic resonance. In an exemplary embodiment of this method, magnetic field gradients maybe added to the electron paramagnetic analysis which allows the imaging and a 3D profiling of the oxygen tension of the tissue.

In yet another aspect of the present invention, a method of manufacturing a microneedle patch for assessing oxygenation at a localized region of interest comprises providing microneedle projections bonded to a layer of material enabling the assembly to be releasably coupled with the localized region of interest, and dispersing a paramagnetic material in the microneedle projections and/or the layer of material.

These and other aspects and advantages of the invention are evident in the description which follows and in the accompanying drawings.

### BRIEF DESCRIPTION OF SEVERAL VIEWS OF THE DRAWINGS

The novel features of the invention are set forth with particularity in the appended claims. A better understanding of the features and advantages of the present invention will be obtained by reference to the following detailed description that sets forth illustrative embodiments, in which the invention are utilized, and the accompanying drawings of which:

FIG. 1 illustrates an exemplary embodiment of a paramagnetic material patch.

FIG. 2 illustrates another exemplary embodiment of a paramagnetic material patch.

FIG. 3 illustrates another exemplary embodiment of a paramagnetic material patch.

FIG. 4 illustrates another exemplary embodiment of a paramagnetic material patch.

### DETAILED DESCRIPTION OF THE INVENTION

Disclosed herein are exemplary embodiments of a novel system and methods for measuring surface oxygen tension in either a human or animal subject. Oxygen tension measurement with the systems and methods described below are based upon the principle of electron paramagnetic resonance (EPR) oximetry. Additional details about EPR are disclosed in references previously incorporated by reference above, as well as in the journal article “Theory, Instrumentation, and Applications of Electron Paramagnetic Resonance Oximetry” by Rizwan Ahmad and Periannan Kuppusamy, published Mar. 10, 2010 in Chem. Rev. 2010,110, pages 3212-3236, the entire contents incorporated herein by reference.

An exemplary system is a microneedle patch which is comprised of microneedle projections which extend into the region of interest, a layer of material bonded to the microneedle projections which enable the assembly to releasably engage the localized region of interest, and a paramagnetic material dispersed within the assembly. An additional layer of material may be disposed over the microneedle projections such that environmental oxygen is inhibited from interacting with the paramagnetic material. The microneedles may be oxygen permeable and composed of materials such as polymethylmethacrylate, polydimethysiloxane, polytetrafluorinated ethylene, SU-8, or silicon. The microneedles may also be hollow. The size of the microneedles can range from 10 microns to 1 millimeter to enable them to cross physiological barriers in tissue. The layer bonded to the microneedles may be composed of the same material as the microneedles or an alternate material. Exemplary materials include polymethylmethacrylate, polyester, polydimethysiloxane, polytetrafluorinated ethylene and polyethylene. This layer may be bonded to the microneedles by chemical or physical means. Suitable paramagnetic probes for EPR oximetry include but are not limited to India ink, coals, char, carbon black, lithium phthalocyanine, lithium naphthalocyanine, nitroxides, or trityl radicals. Paramagnetic probe particles can range in size from 100 nm to 1 mm particle diameter to enable ease of integration into the microneedle patch. An exemplary method includes application of the microneedle patch to a subject that will releasably engage a localized region of interest to determine surface oxygen tension using a suitable EPR spectrometer for oximetry under normobaric and/or hyperbaric conditions. Use of this method will enable accurate measurement across physiological barriers in the tissue and allow accurate assessment of in vivo oxygenation. The method may also comprise application of the microneedle patch to determine and identify tissue hypoxia based upon measurement of oxygen tension using EPR oximetry. Individual or multiple magnetic field gradients can also be applied as part of this method to enable mapping of oxygenation in tissue in 1-, 2-, or 3-dimensions. Fast fourier transform (FFT) can also be used as part of this method to analyze the signal from application of individual or multiple magnetic field gradients. Another exemplary method involves manufacturing a microneedle patch by providing microneedle projections bonded to a layer of material adapted to be releasably coupled with a localized region of interest; and dispersing a paramagnetic material in the microneedle projections and/or the layer of material.

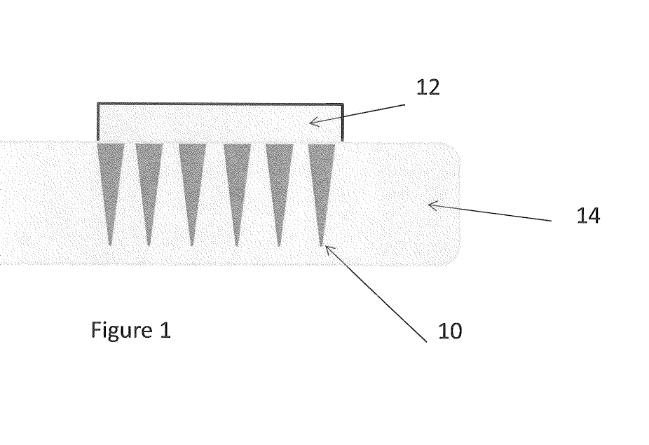

FIG. 1 illustrates an exemplary embodiment of the system for measuring surface oxygen tension. Suitable paramagnetic materials for EPR oximetry such as those discussed above are embedded or dispersed within the microneedle projections 10, and a layer of material 12 is bonded to the microneedles to enable attachment and release from a tissue of interest. Upon application to the tissue, oxygen from the skin 14 diffuses into the microneedles and interacts with the paramagnetic material. This can be excited by an EPR spectrometer to determine oxygen tension, such as the handheld scanner disclosed in U.S. patent application Ser. No. 13/481,545, the entire contents of which are incorporated herein by reference.

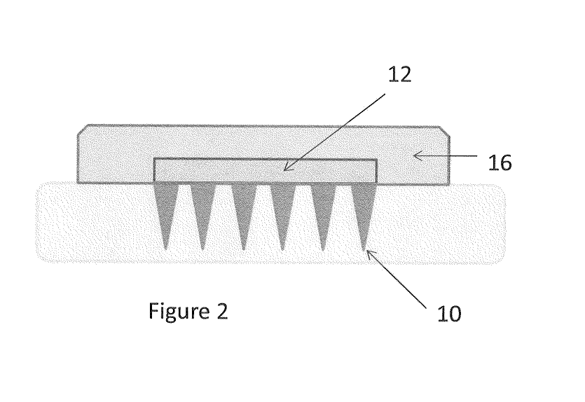

FIG. 2 illustrates an alternative embodiment similar to FIG. 1, except in this embodiment there is there is an additional layer of material 16 over the microneedle patch to inhibit environmental oxygen from interacting with the paramagnetic materials.

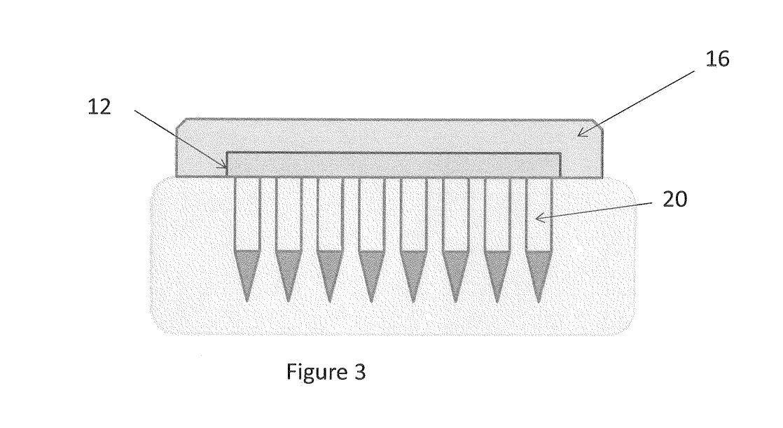

FIG. 3 illustrates a third possible construction method of the microneedles 20. The microneedles 20 may be cylindrical with conical tips 22, or they may be rectangular or square in cross section. The needles may be hollow and filled with the EPR sensitive material such as a crystal in a PDMS or some polymer and may be filled or impregnated with the material to different depths. The needles may be hollow filled with the EPR sensitive material in a silicone matrix. In either case, the EPR sensitive material may fill only a section of the needle or the entire needle. The needles may also be square or rectangular in cross section.

The needles may be made in varying lengths thus interrogating the tissue 14 to varying depths.

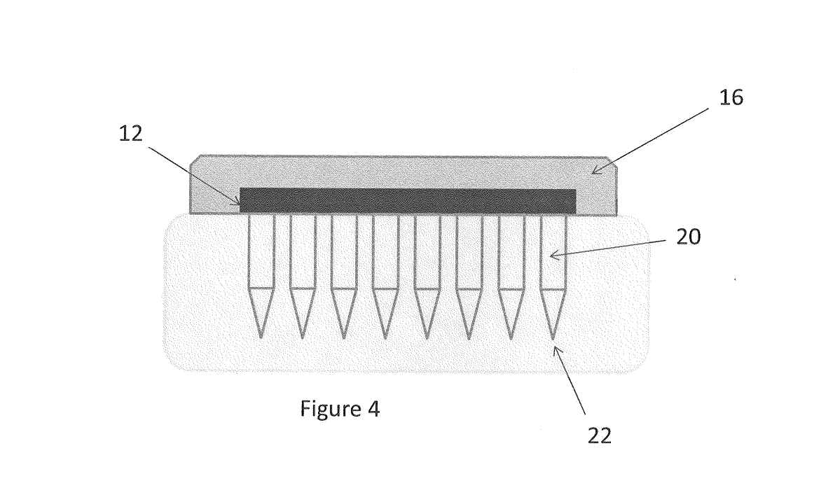

As shown in FIG. 4, the microneedles may also be hollow with a hole at the tip r holes along the length of the needle to allow gas and O₂ permeability. This allows the EPR sensitive substrate above the skin to equilibrate to the O₂ of the tissue that the needle penetrates to.

While preferred embodiments of the have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A microneedle patch for assessing oxygenation at a localized region of interest, said patch comprising:
a plurality of microneedle projections which extend into the localized region of interest;
a first layer of material chemically or physically bonded to the microneedle projections which enable the assembly to releasably engage the localized region of interest; and
a paramagnetic material.

2. The patch of claim 1, comprising a second layer of material disposed over the microneedle projections such that environmental oxygen is inhibited from interacting with the paramagnetic material.

3. The patch of claim 1, wherein the microneedle projections comprises an oxygen permeable material.

4. The patch of claim 1 wherein the paramagnetic material is contained within the first microneedle projections.

5. That patch of claim 1 wherein the paramagnetic material is contained within the layer bonded to the microneedle projections.

6. The patch of claim 1, wherein the microneedle projections are hollow.

7. The patch of claim 1, wherein the microneedle projections comprises polydimethylsiloxane or derivatives thereof.

8. The patch of claim 1, wherein the microneedle projections comprises polymethylmethacrylate or derivatives thereof.

9. The patch of claim 1, wherein the microneedle projections comprises SU-8 or derivatives thereof.

10. The patch of claim 1, wherein the microneedle projections comprises silicon.

11. The patch of claim 1, wherein the microneedle projections are between 10 micrometers and 1 millimeter in height.

12. The patch of claim 1, wherein the paramagnetic material comprises a lithium phthalocyanine derivative.

13. The patch of claim 1, wherein the paramagnetic material comprises lithium octa-n-butoxy-naphthalocyanine or a derivative thereof.

14. The patch of claim 1, wherein the paramagnetic material comprises India ink.

15. The patch of claim 1, wherein the paramagnetic material consists of paramagnetic particles ranging from 100 nanometers to 1 mm in size.

16. A method for assessing oxygenation at a localized region of interest, said method comprising:
providing a microneedle patch having paramagnetic material dispersed in a layer thereof;
releasably coupling the patch with the localized region of interest; and
assessing oxygenation at the localized region of interest using electron paramagnetic resonance.

17. A method of claim 16, also comprising inhibiting environmental oxygen from interacting with the paramagnetic material.

18. A method of claim 16, wherein magnetic field gradients are used as part of the electron paramagnetic resonance analysis to do a 3D mapping of the oxygen tension of the tissue.

19. A method of claim 18, wherein the magnetic field gradient is applied in only one dimension and a projection profile of the O₂ tension obtained;

20. A method of claim 18, wherein multiple magnetic field gradients are applied in more than one dimension to obtain a detailed 1D, 2D or 3D mapping of O₂ tension measured by the paramagnetic crystals in the microneedle patch.

21. A method of claim 18, wherein the magnetic field gradients may be applied as simple gradients to get projection images or be used in a phase encoding manner to get small pixel resolution.

22. A method of claim 18, wherein fast fourier transform is used on the electron paramagnetic resonance signal obtained by the use of magnetic field gradients to deconvolve the data into voxels.

23. A method of manufacturing a microneedle patch for assessing oxygenation at a localized region of interest, said method comprising:
providing microneedle projections bonded to a layer of material adapted to be releasably coupled with the localized region of interest; and
dispersing a paramagnetic material in the microneedle projections and/or the layer of material.

24. A method of claim 23, also comprising coupling a layer of material with the microneedle projections which inhibits environmental oxygen from interacting with the paramagnetic material.

25. A method of claim 23, wherein the paramagnetic material is first dispersed into a suitable polymer material which is cast and/or molded into microneedle projections.

26. A method of claim 23, wherein the microneedle projections are patterned/cast from the same material as the layer bonded to it.
